(19) United States
(12) Patent Application Publication
(10) Pub. No.: US 2007/0161949 A1
(43) Pub. Date: Jul. 12, 2007

(54) Catheter system for minimizing retrograde bacterial transmission from a catheter tubing

(76) Inventors: Susan Jane Knox, Stanford, CA (US); Caroline E. Schore, Davis, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/327,835

(22) Filed: Jan. 6, 2006

(51) Int. Cl. A61M 31/00; A61M 37/00

(52) U.S. Cl. 604/93.01

(57) ABSTRACT

The present invention provides systems and methods for minimizing the retrograde transmission from the catheter tubing. The invention is particularly suitable for reducing the likelihood of a patient acquiring a urinary tract infection associated with an indwelling catheter. The systems and methods include a catheter tubing having a proximal and distal end with a filter located therebetween and a means for accessing negative pressure for creating a pressure differential between the proximal and distal ends of the catheter tubing.

### FIELD OF THE INVENTION

The present invention relates to catheter systems and methods for minimizing retrograde bacterial transmission from a catheter tubing. The subject systems and methods reduce the likelihood of a patient acquiring a catheter associated bacterial infection. In particular, the present invention is useful in reducing the incidence of urinary tract infections related to indwelling urinary catheters.

### BACKGROUND OF THE INVENTION

Catheters are commonly inserted into canals, vessels, passageways, or body cavities, in order to allow injection or withdrawal of fluids from a patient. It is well known that catheters may lead to serious infections by introducing microorganisms into a patient. The infection may occur when inserting the catheter into a patient or may ascend upwardly along the exterior surfaces of the catheter over a period of time. As such, the likelihood of infection increases the longer the catheter is in place. Examples of catheters susceptible to bacterial infection include central venous catheters, vascular catheters, peripherally inserted catheters, endotracheal catheter and urinary catheters.

In fact, the most common nosocomial infection in hospitals and elderly care facilities are urinary tract infections related to indwelling catheters. The urinary tract infections are usually harmless, however, persisting infections may result in prostatitis, epididymitis, cystitis, pyelonephritis, and gram-negative bacteremia, particularly in high-risk patients.

A variety of pathogens have been identified as the cause of infections related to indwelling catheters, including Escherichia coli, Klebsiella, Proteus, Pseudomonas, Enterobacter, Serratioa, and Candida. These microorganisms are either part of the patient's natural flora, acquired by cross-contamination from other patients or hospital personnel or acquired by exposure to contaminated solutions or non-sterile equipment.

The development of the closed drainage system significantly reduced the number of infections resulting from the reflux of the infective agent through the catheter tubing. However, sterile closed drainage systems proved difficult to maintain and easily leaked as a result of ineffective sealing elements.

Recent efforts have been made to improve the efficiency of the closed drainage system, such as the addition of a sampling port in the drainage tubing or the preconnected catheter tube system; both of which prevent the opening of the closed system. Other advances have included the insertion of air vents, drip chambers, and one-way valves that were designed to prevent the reflux of contaminated fluid. Additional attempts to reduce bacterial infections include coating the catheter with an antimicrobial agent. Although these modifications have offered some improvement, none have significantly reduced the frequency of bacterial infections associated with catheters. Moreover, these additional components increase the complexity of the catheter systems which may lead to improper assembly.

As such, there is continued interest in the identification of new methods for minimizing the number of catheter related bacterial infections. Of particular interest would be the development of methods that are relatively inexpensive, uncomplicated, and applicable to already existing catheter systems.

### SUMMARY OF THE INVENTION

The present invention provides an improved catheter system which minimizes infections associated with using a catheter.

The systems of the present invention are particularly suitable for minimizing the risk of urinary tract infections in patients with urinary catheters.

The catheter system of the present invention includes a catheter tubing having a proximal end and a distal end, at least one filter therebetween, and a means for accessing negative pressure for creating a pressure associated with the distal end of the catheter tubing. The pressure created at the distal end of the catheter tubing results in a pressure differential between the proximal end and the distal end of the catheter tubing wherein the pressure at the distal end is less than the pressure at the proximal end. This pressure differential minimizes bacterial infections by reducing retrograde bacterial transmission from the catheter tubing into the patient.

The filter positioned between the proximal and distal ends of the catheter tubing may be a separate inserted filter or integrated within the catheter tubing.

The catheter system of the present invention may further include a fluid collection container located at the proximal end of the catheter tubing.

The negative pressure means may be connected to a fluid collection container, thereby creating a negative pressure within the container itself.

The catheter system may further include an antimicrobial agent which either coats the inside or outside of the catheter tubing. The antimicrobial agent may additionally be integrated within the filter.

The system of the present invention may further include at least one valve for controlling the fluid flow or air flow within the catheter tubing.

The catheter system of the present invention may further be connected to a fluid collection container.

The methods of the present invention minimize a patient's risk of acquiring a bacterial infection associated with a catheter. One method includes providing a patient with a catheter having a proximal end and a distal end with a filter positioned therebetween. A negative pressure is accessed and associated with the distal end of the catheter tubing. A pressure differential is created wherein the distal end has a pressure less than the proximal end of the catheter tubing. The pressure differential prevents retrograde bacterial transmission thereby minimizing the likelihood of infecting the patient.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Also for purposes of clarity, certain features of the invention may not be depicted in some of the drawings. Included in the drawings are the following figures:

FIG. 1A is a schematic representation of the catheter system of the present invention comprising a catheter tubing having a proximal and distal end with a filter inserted therebetween.

FIG. 1B is a schematic representation of a filter, which may be inserted into the proximal end of the catheter tubing.

FIG. 2 is a schematic representation of the catheter system of the present invention comprising a catheter tubing having a proximal and distal end with a filter integrated within the catheter tubing

### DETAILED DESCRIPTION OF THE INVENTION

Before the systems and methods of the present invention are described, it is to be understood that this invention is not limited to particular therapeutic applications and implant sites described, as such may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. The terms “proximal” and “distal” when used to refer to the catheter tubing of the present invention are to be understood to indicate positions or locations relative to the user where proximal refers to a position or location closer to the user and distal refers to a position or location farther away from the user.

It must be noted that as used herein and in the appended claims, the singular forms “a”, “an”, and “the” include plural referents unless the context clearly dictates otherwise. Thus, for example, reference to “a valve” may include a plurality of such valves and reference to “an antimicrobial agent” includes reference to one or more antimicrobial agents and equivalents thereof known to those skilled in the art, and so forth.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The present invention will now be described in greater detail by way of the following description of exemplary embodiments and variations of systems and methods of the present invention. While certain particular applications (e.g., urinary catheters) are referenced in the following description, this is not intended to be limiting as the devices, systems and methods of the present invention may be employed with any appropriate catheter application, including but not limited to, central line catheters, vascular catheters, peripherally inserted catheters, endotracheal catheter and urinary catheters.

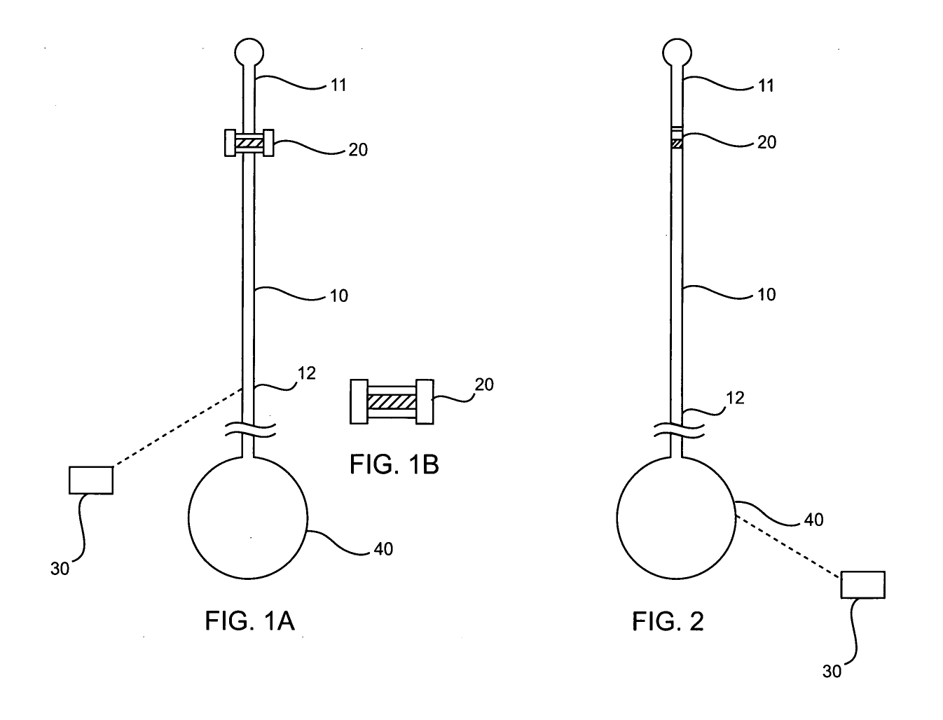

As summarized above, the present invention provides an improved catheter system which minimizes infections associated with using a catheter. The systems of the present invention are particularly suitable for minimizing the risk of urinary tract infections in patients with urinary catheters. Referring now to FIGS. 1A and 2 in particular, these are illustrated exemplary embodiments of the catheter systems of the present invention. Each of the systems have a catheter tubing (10) having a proximal end (11) and a distal end (12) with a filter (20) positioned therebetween. The filter (20) positioned between the proximal (11) and distal ends (12) of the catheter tubing may be a separate inserted filter as in FIG. 1B or integrated within the catheter tubing as in FIG. 2. The system further includes a means for accessing negative pressure for creating a pressure associated with the distal end (12) of the catheter tubing (30). The pressure created at the distal end (12) of the catheter tubing (10) results in a pressure differential between the proximal end (11) and the distal end (12) of the catheter tubing (10) wherein the pressure at the distal end (12) is less than the pressure at the proximal end (11). The created pressure differential between the proximal (11) and distal ends (12) of the catheter tubing (10) prevents retrograde transmission, thereby minimizing the likelihood that a patient will acquire an infection from the catheter tubing (10). The catheter system may further be connected to a fluid collection container (40). The negative pressure means (30) may be connected with the catheter tubing (10) at its distal end (12) as in FIG. 1 or may be directly connected to the fluid collection container (40) as in FIG. 2. The subject systems may also include an antimicrobial agent coating on the outside or inside of the catheter tubing or integrated within the filter. The subject systems may further include at least one valve positioned along the catheter tubing.

The catheter tubing of the subject catheter system may be constructed from a biocompatible thermoplastic material such as Thermoplastic PolyUrethanes. One of skill in the art will appreciate that the subject catheter tubing may be constructed from any suitable flexible material, for example, PolyEther Block Amide, PolyOlefins, PolyVinyl Chloride, PolyVinylidene Fluoride, and Styrene-Ethylene-Butylene Styrene Block Copolymer.

As shown in FIGS. 1A and 2, the system further includes a means for accessing negative pressure (30) for creating a pressure associated with the distal end (12) of the catheter tubing (10). Alternatively, the fluid collection device could be made to be under negative pressure. For example, prior to packaging, the drainage bag may be suctioned to create a negative pressure within the bag, or a simple bulb or small vacuum device (e.g., pump) that can be regulated could be incorporated into the collection device. Still yet, the catheter tubing itself may be under differential pressure. Several types of catheters having differential pressure are currently available, including chest tubes, surgical drainage tubes and intraventricular drains used for the brain. As gravity alone creates some differential in pressure, some circumstances no additional mechanism for creating a pressure differential may be needed.

In any of these embodiments, the pressure created at the distal end (12) of the catheter tubing (10) results in a pressure differential between the proximal (11) end and the distal end (12) of the catheter tubing (10) wherein the pressure at the distal end is less than the pressure at the proximal end. The created pressure differential between the proximal and distal ends of the catheter tubing prevents retrograde bacterial transmission, thereby minimizing the likelihood that a patient will acquire a bacterial infection from the catheter tubing. However, other embodiments of the present invention may be suitable without this pressure differential

The pump may, for example, be electric or any other suitable pump. Examples of an electric pump include various types of pumps such as a piston pump, a plunger pump, and a wing pump. In particular, a diaphragm pump is preferably used. The diaphragm pump has a characteristic that a pump chamber is not easily contaminated, and is relative compact and light and has a long durable life and consumes less power. In the diaphragm pump, one side of a pump chamber is constituted by an elastic film made of rubber or plastics and this elastic film is moved by increasing or decreasing a pressure of air or an amount of liquid from its back or is moved mechanically or electrically. A mechanical vacuum gauge may also be incorporated for observing the amount of pressure created by the negative pressure means.

The pressure pump may be further connected to a microprocessor/controller pressure within the catheter tubing and fluid container. The microprocessor may be any hardware or software which performs the functions of controlling the pump in order to continually maintain a negative pressure associated with the distal end of the catheter tubing. An exemplary embodiment of a microprocessor to be used with the subject invention is a programmable digital microprocessor such as a mainframe, server, or personal computer. Where the processor is programmable, suitable programming can be communicated from a remote location to the processor, or previously saved in a computer program product, for example, a portable or fixed computer readable storage medium, whether magnetic, optical or solid state device based.

The subject systems further include a filter positioned between the proximal end and the distal end of the catheter tubing. The filter is used to prevent the passage of microorganisms up through the catheter tubing especially from the distal end of the catheter tubing. The filter may be located at various positions between the proximal and distal ends of the catheter tubing. For example, the filter (20) in FIG. 1A is positioned adjacent to the very proximal end (11) of the catheter tubing (10) wherein the filter (20) in FIG. 2 is positioned further down, almost midway between the distal (12) and proximal (11) ends of the catheter tubing. In certain embodiments, more than one filter may be positioned at various locations between the proximal and distal ends of the catheter tubing.

In the embodiment in FIG. 1A, the filter (20) is a separate component as shown in FIG. 1B, which is inserted into the catheter tubing (10). Because the filter (20) is not incorporated into the catheter tubing, one may easily remove and replace the filter. However, in another embodiment of the present invention; the filter (20) may be integrated within the catheter tubing as shown in FIG. 2.

One of skill in the art would fully understand that any filter capable of trapping microorganisms may be utilized in the subject invention. U.S. Pat. No. 6,852,224, herein incorporated by reference, is drawn to a filter comprising activated carbon fibers, which has a Virus Removal Index of at least about 99%. U.S. Pat. No. 5,714,343, herein incorporated by reference, discloses absorbing pads surmounted by a retaining membrane. The fluid is passed through the retaining membrane and the microorganisms potentially retained on the membrane are visualized by a chromogenic agent having an oxidation potential. U.S. Pat. No. 4,828,698, herein incorporated by reference, teaches the use of a microporous membrane having pore sizes from 0.02 μm to 0.05 μm for microbiological control. Further examples of filters that may be used with the present invention are acrodiscs for syringes, filters for tissue culture medium, vacushields for tissue culture hoods, and Porex hydrophobic pipette filters. It is further appreciated that the filters in the subject system may be disposable for easy replacement. One of skill in the art would readily understand that the above filters are exemplary and not to be construed as a limitation of the subject catheter systems.

The catheter systems discussed above may also be connected to a fluid collection container (40) as shown in FIGS. 1A and 2. The fluid collection container (40) would be connected to the distal end (12) of the catheter tubing (10). The container is preferably constructed of polyethylene or polypropylene but may be made of other impervious or resilient materials. The fluid collection container may further include at least one filter and at least one antimicrobial agent. Any of the various collection devices well known in the art may be used with the catheter systems of the present invention.

The negative pressure source (30) may be directly connected to the fluid container (40) as in FIG. 2. In this embodiment, a small tube may be employed to connect the container to a small pump and pressure switch or vacuum transducer and switch. The pump evacuates air from the container until it reaches a set vacuum at which the pressure switch or vacuum transducer and switch, turns off the pump. The pressure switch, or vacuum and transducer and switch, is set so that every time the vacuum falls slightly below the set vacuum, the pump will activate again. By this means a vacuum is maintained in the container (40). The fluid container (40) may also include an additional filter between the pump and the opening of the fluid container (40) to prevent any liquid from being pumped out of the container.

In some embodiments, the antimicrobial agent may be directly integrated within the filter. In some instances, the antimicrobial agent will be added to a resin prior to applying it to the catheter system. Examples of suitable resins include styrene-butadiene rubbers, polyurethanes, silicone polyurethanes, polyvinylchloride, polyolefin, elastomers, and silicone. Other materials such as an inorganic or organic hybrid material, carbon and other high area filled materials such as nonocomposites, hydrogels such as polyoxazoline, polyvinylalcohol, polyhydroxy acrylates, super absorbent polymers and biodegradable and natural polymeric materials such as cellulose or sponges, can also be used as carriers for the antimicrobial composition integrated within the filter.

In other embodiments, the antimicrobial agent coats the inside of the catheter tubing and may also coat the outside of the catheter tubing at its proximal end. Antimicrobial agents useful with the present invention include but are not limited to the biguanides, especially chlorhexidine and its salts, including chlorhexidine acetate, chlorhexidine gluconate, chlorhexidine hydrochloride, and chlorhexidine sulfate, silver and its salts, including silver iodate, silver iodide, silver lactate, silver laurate, silver nitrate, silver oxide, silver palmitate, silver protein, and silver sulfadiazine, polymyxin, tetracycline, aminoglycosides, such as tobramycin and gentamicin, rifampicin, bactracin, neomycin, chloramphenicol, miconazole, quinolones such as oxolinic acid, norloxacin, nalidixic acid, pefloxacin, enoxacin and ciprofloxacin, penicillins such as oxacillin and pipracil, nonoxynol, fusidic acid, cephalosporins, and combinations thereof.

Instead of an antimicrobial agent, the catheter system may also include silver and zinc concentrations. When the silver and zinc concentrations contact bodily fluids, an electrical current arises between the silver and zinc concentrations. This current attracts microbes to the silver concentrations wherein upon contact, the microorganisms are killed. In other words, all pathogenic organisms carry a negative charge. When the bimetallic pattern of dissimilar metals is moistened by body fluids, a current flow is induced that draws the bacteria and other pathogenic organisms to the positively charged silver anode. The silver attaches to the sulfhydryl groups and denatures the protein complexes vial to respiration and other functions for survival, thereby killing the organisms.

The catheter systems of the present invention may further include at least one valve but may include multiple valves at various positions in the catheter system. One of skill in the art would fully appreciate that the valve may be any assembly capable of integral attachment to the catheter tubing. The valve is preferably a two-way valve that allows fluid to flow in either direction through the valve. In a preferred embodiment, the valve may be a positive displacement, luer-activated valve designed to prevent fluid from being drawn back into the catheter tubing. The valve also avoids potential clogging of the catheter tubing which also assists in preventing bacterial contamination of the catheter tubing.

The valve of the subject catheter systems may be manufactured of any acceptable medical grade plastic which is capable of being precision molded and adapted to maintain its dimensions under everyday conditions. Preferably the valve is prepared from a hard plastic, but it may be made of other medically inert materials known to those of ordinary skill in the art. When employing a positive displacement valve, the material of the valve is preferably resistant to alcohol and has a low coefficient of friction. Examples of suitable materials include polycarbonate, PVC, nylon, Delrin, and hydrel. In a preferred embodiment, the valve is constructed from polycarbonate for its ability to be sterilized.

In one embodiment, the catheter system may also include a housing located where the distal end of the catheter tubing connects to the fluid container. The housing is capable of dispensing an antimicrobial agent into the catheter tubing and/or the fluid container. For example, the housing may comprise an adaptor for connecting the distal end of the catheter with a connector on a fluid collection bag. The adaptor also includes a chamber for housing an antimicrobial agent. A valve is included in the flow passage and has a first position where the flow passage is open and a second position where the flow passage is closed. The valve receives a first dose of antimicrobial agent when the valve is closed and disperses the antimicrobial agent back into the flow path between the indwelling catheter and the collection bag.

The catheter system of the present invention may also include a sensor or sensors to monitor pressure, flow, volume, and fluid leakage. The sensor may be directly attached to the catheter tubing or may be included within the fluid collection container or secured within the filter of the catheter tubing of the present invention. The sensor may further employ a wireless means to deliver information from the implantation site to an instrument external to the body.

One method of the subject invention is a method for minimizing retrograde transmission from a catheter tubing in a patient by using the subject systems described above. The method includes: providing a catheter having a tubing having a proximal end and a distal end with at least one filter positioned therebetween and a means for accessing negative pressure. The method further comprises applying negative pressure to the catheter tubing, thereby creating a pressure differential between the proximal and distal ends. Additionally, the method may further include connecting a fluid container to the catheter tubing and draining a fluid from the patient.

Also provided by the subject invention are kits for use in practicing the subject methods. The kits of one embodiment of the subject invention includes catheter tubing and at least one filter and at least one negative pressure means, as described above. The kits may also include one or more antimicrobial agents for use with the subject invention. The kits may additionally include a fluid collection container. Other kits include at least one negative pressure source and at least one valve. Finally, the kits may further include instructions for using the subject catheter systems.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A catheter system for minimizing retrograde bacterial transmission from a catheter tubing, said system comprising:
a catheter tubing comprising a proximal end and a distal end;
at least one filter positioned between said proximal end and said distal end of said catheter tubing; and
a means for accessing negative pressure for creating a pressure differential between the proximal and distal ends of the catheter tubing.

2. The system of claim 1 wherein a pressure at said distal end of said catheter tubing is less than a pressure at said proximal end.

3. The system of claim 1 further comprising a fluid collection container.

4. The system of claim 1 wherein said catheter is one of a urinary catheter, a central line catheter, a vascular catheter, a peripherally inserted catheter and an endotracheal catheter.

5. The system of claim 1 further comprising an antimicrobial agent.

6. The system of claim 5 wherein said antimicrobial agent is integrated within said filter.

7. The system of claim 5 wherein said antimicrobial agent coats the inside of said catheter tubing.

8. The system of claim 5 wherein said antimicrobial agent coats at least the outside of said catheter tubing.

9. The system of claim 5 wherein said antimicrobial agent coats the outside and inside of said catheter tubing.

10. The system of claim 3 further comprising an antimicrobial agent in said fluid collection container.

11. The system of claim 1 further comprising an adapter positioned in between said distal end of said catheter tubing and said opening of said fluid collection container.

12. The system of claim 1 wherein said adaptor includes a chamber containing an antimicrobial agent.

13. The system of claim 1 wherein the source of the negative pressure means is a pump.

14. The system of claim 13 wherein said pump is connected to a microprocessor.

15. The system of claim 1 further comprising at least one valve.

16. The system of claim 1 further comprising at least one sensor.

17. A catheter system for minimizing retrograde bacterial transmission from a catheter tubing, said system comprising:
a catheter tubing comprising a proximal end and a distal end wherein said distal end has a lower pressure than said proximal end; and
at least one filter positioned between said proximal end and said distal end of said catheter tubing.

18. The system of claim 17 further consisting of a fluid collection container.

19. The system of claim 18 wherein a negative pressure is created within said distal end of said catheter tubing and within said fluid collection container.

20. The system of claim 17 further comprising an antimicrobial agent.

21. The system of claim 20 wherein the antimicrobial agent coats the inside or outside of said catheter tubing.

22. The system of claim 19 further comprising a negative pressure source chosen from the group consisting of a vacuum, a diaphragm, and a balloon.

23. The system of claim 17 further comprising an adapter positioned in between said distal end of said catheter tubing and said opening of said fluid collection container.

24. The system of claim 17 wherein said adaptor includes a chamber containing an antimicrobial agent.

25. The system of claim 17 further comprising at least one sensor.

26. A method of minimizing retrograde bacterial transmission from a catheter tubing in a patient, said method comprising:
providing a catheter tubing comprising:
a proximal end and a distal end,
at least one filter positioned between said proximal and distal ends, and
a means for accessing a negative pressure; and
creating a pressure differential between said proximal and distal ends of said catheter tubing.

27. The method according to claim 26 further comprising: connecting said means for accessing negative pressure to a source providing negative pressure.

28. The method according of claim 26 further comprising:
connecting said catheter tubing to a fluid container; and
draining a fluid from said patient.

29. The method according to claim 28 further comprising introducing an antimicrobial agent into said fluid container.

30. A kit for use in minimizing the retrograde bacterial transmission from a catheter tubing, said kit comprising:
a catheter tubing;
at least one filter;
a negative pressure means; and
instructions for practicing the method of claim 26.

31. The kit according to claim 30 further comprising at least one valve.

32. The kit according to claim 30 further comprising at least one sensor.

33. The kit according to claim 30 further comprising a fluid collection container.

34. The kit according to claim 30 further comprising at least one antimicrobial agent.
